(19) United States
(12) Patent Application Publication
(10) Pub. No.: US 2007/0173736 A1
(43) Pub. Date: Jul. 26, 2007

(54) Apparatus and methods for endometrial biopsies

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSPEC LLC, San Francisco, CA (US)

(21) Appl. No.: 11/492,501

(22) Filed: Jul. 24, 2006

Related U.S. Application Data
(63) Continuation-in-part of application No. 11/245,498, filed on Oct. 7, 2005.

(51) Int. Cl. A61B 10/00

(52) U.S. Cl. 600/562; 600/570; 600/572; 600/563

(57) ABSTRACT

Apparatus and methods for endometrial biopsies, wherein an applicator is disposed on the distal end of an outer tube and an injection device is connected to the proximal end of the same tube. The applicator comprises one or more ridges on its outer surface that anchor the applicator in the cervical os, and that cause the applicator to remain securely positioned and maintain the os in a dilated position during the entire procedure. The ridges may be shaped like protrusions, scalloped edges or grooves, and may be disposed in a helical or circular pattern. A method for performing endometrial biopsies is also provided. The apparatus and methods according to the present invention are configured to minimize discomfort to the patient while facilitating the operation of the clinician.

### RELATED APPLICATIONS

This application is a continuation-in-part of U.S. patent application Ser. No. 11/245,498, filed on Oct. 7, 2005.

### FIELD OF THE INVENTION

The present invention relates to apparatus and methods for performing endometrial biopsies. More particularly, the present invention relates to apparatus and methods for applying an anesthetic and removing uterine cells that provide the clinician with improved flexibility while at the same time reducing discomfort to the patient.

### BACKGROUND OF THE INVENTION

Endometrial biopsies are procedures employed for evaluating uterine tissue for the presence of cancerous or precancerous cells. Most women undergoing this procedure suffer from abnormal uterine bleedings or from amenorrhea, a prolonged absence of the menstrual period. Other women undergoing this procedure include women with a high risk of endometrial cancer such as hereditary colorectal cancer, or with infertility problems.

Endometrial biopsies essentially involve the insertion of a catheter through the cervix and into the uterus of the patient, and the aspiration of a small amount of endometrial lining into the catheter. These procedures are typically performed in a clinician's office and have proven to provide tissue samples that are equal or superior to the previous dilation and curettage procedures.

Unfortunately, endometrial biopsies can be highly uncomfortable to the patient, in spite of the use of anesthetics. The placement of the catheter through the cervix and inside the uterine cavity can produce cramps, in spite of the use of painkillers prior to the procedure and of the spraying with a numbing medication during the procedure. Further, the clinician often must twirl the catheter while moving it in and out of the uterine cavity, to enhance uptake of the uterine tissue. The activity of the clinician can be complicated by the effort to maintain a proper positioning of the catheter inside the uterus during the procedure, in spite of constrictive actions of the cervical os and of movements of the patient's body.

Endometrial biopsies in the prior art typically involve the application of a plastic acorn against the cervical os, which is disposed at the distal end of an elongated tube. A syringe containing an anesthetic is connected to the proximal end of the tube, and an anesthetic is released into the uterine cavity. A catheter is successively inserted through the tube and into the uterine cavity.

One disadvantage of endometrial biopsies in the prior art is that some leakage of anesthetic may occur due to an imperfect contact of the acorn with the cervical os, reducing the duration and extent of the relief from pain that is available during the extraction of tissue samples.

Another disadvantage of endometrial biopsies in the prior art is that multiple devices must be inserted serially into the uterus to obtain the desired sample, increasing the complexity of the procedure and the discomfort to the patient. For example, in U.S. Pat. No. 6,423,038 to Vancaillie, an apparatus for intrauterine anesthesia is described, which is used to administer anesthetic and which is then withdrawn to allow access for another instrument.

A further disadvantage of endometrial biopsies in the prior art is the level of difficulty in maintaining the cervical os in a dilated position during the procedure. Clinicians tend to resolve this dilation problem by maintaining the cervical os in an open position with the use of an additional instrument, typically a tenaculum.

In view of the foregoing, it would be desirable to provide apparatus and methods for endometrial biopsies, in which the acorn is securely retained in the cervical os during application of the anesthetic.

It also would be desirable to provide apparatus and methods for endometrial biopsies that reduce the number of instruments that must be separately inserted into and removed from the uterine cavity.

It further would be desirable to provide apparatus and methods for endometrial biopsies that enable a clinician to retain the cervical os in a dilated position without the use of supplemental instruments.

### SUMMARY OF THE INVENTION

In view of the foregoing, it is an object of the present invention to provide apparatus and methods for endometrial biopsies that overcome the drawbacks of the prior art and that increase the operating flexibility of the clinician while reducing patient discomfort.

It is also an object of the present invention to provide apparatus and methods for endometrial biopsies that enable a secure retention of the plastic acorn in the cervical os during application of the anesthetic.

It is another object of the present invention to provide apparatus and methods for endometrial biopsies that enable a clinician to retain the cervical os in a dilated position without the use of supplemental devices.

It is a further object of the present invention to provide apparatus and methods for endometrial biopsies that reduce the number of instruments that must be separately inserted and removed from the uterine cavity.

These and other objects of the present invention are accomplished by providing apparatus and methods for endometrial biopsies having an applicator disposed on the distal end of an outer tube and an injection device connected to the proximal end of the same tube. The applicator is configured to be securely positioned in the cervical os and to maintain the os in a dilated position during the injection of an anesthetic medication and the later insertion of a tissue-removing catheter. The apparatus and methods of the present invention is configured to minimize discomfort while facilitating the operation of the clinician.

In the first embodiment, the outer tube has a length of 20-30 cm and a diameter of 3-5 mm, and is preferably manufactured from plastic material. A frustoconical applicator, preferably of a silicone material, surrounds the distal end of the outer tube and has one or more grooves disposed on the conical wall in a helical pattern, giving the applicator a screw-like pattern that provides for a tight fit with the cervical os and prevents the undesired release of the applicator. The cervical os is then retained in the desired dilated condition by a secure anchoring of the applicator grooves into the os walls, facilitating the activity of the clinician.

A standard Luer-Lok fitting is attached to the proximal end of the outer tube, enabling the connection with a syringe for the initial injection of an anesthetic and the later insertion of a catheter.

A suction device, typically a syringe, is connected to the proximal end of the catheter, while one or more apertures are provided at the distal end of the catheter. When vacuum is drawn through the suction device, cells lining the uterine walls are drawn into the catheter through the apertures and into the suction device, and can later then be analyzed to determine possible malignant conditions.

In different embodiments, the ridges on the applicator may be shaped like ridges extending from the frustoconical wall, or like layers of varying height on the frustoconical wall. Further, the ridges may be arranged in a helical pattern or be disposed in a parallel circular pattern.

Methods of using the apparatus of the present invention to perform endometrial biopsies also are provided.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view depicting an exemplary embodiment of the apparatus of the present invention;

FIG. 2 is a side view of the individual components of the apparatus of FIG. 1;

FIGS. 3A-3B are side views of applicator 12 in FIG. 1, illustrating different dispositions of the ridges on the wall of applicator 12; and

FIGS. 4A-4C depict alternative shapes of the ridges disposed on the applicators of FIGS. 3A-3B.

### DETAILED DESCRIPTION OF THE INVENTION

The present invention is directed to apparatus and methods for endometrial biopsies that increase the flexibility of operation of a clinician while reducing patient discomfort. In a preferred embodiment, an apparatus is provided that includes an applicator, through which a catheter is inserted and an anesthetic is injected. Grooves on the outer surface of the applicator enable the clinician to maintain the applicator in the cervical os in a secure position and also to maintain the cervical os in a dilated position during the entire procedure.

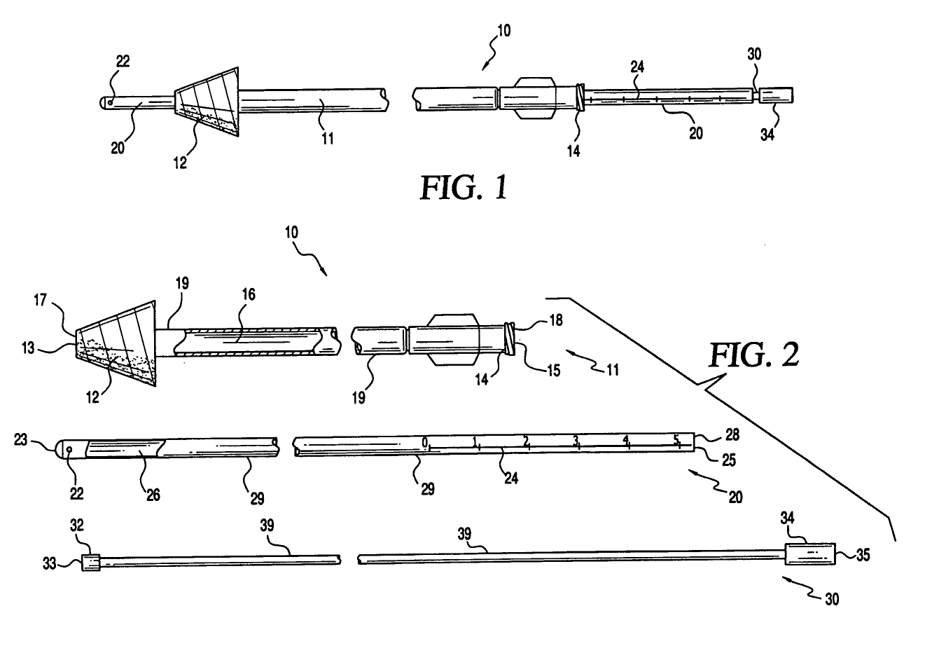

Referring to FIGS. 1 and 2, an exemplary embodiment of an apparatus for endometrial biopsies constructed in accordance with the principles of the present invention is described. Apparatus 10 includes four basic members, outer tube 11, applicator 12, inner tube 20, and injection device 30. Applicator 12 is positioned at distal end 13 of outer tube 11, and Luer-Lok fitting 14 disposed at proximal end 15 of outer tube 11. Lumen 16 extends from distal end 13 through applicator 12 and to proximal end 15 to provide fluid communication between opening 18, at the proximal end of Luer-Lok fitting 14, and opening 17, at the distal end of outer tube 11.

Outer tube 11 is preferably cylindrical and manufactured from a semi-rigid plastic material, such as a silicone, polyethylene, or polycarbonate material, that are sufficiently strong to allow outer tube 11 to retain its tubular shape without wrinkling when applicator 12 is inserted in the cervical os. The selection of a clear plastic material as opposed to a metal or glass material also provides a tube with improved flexibility and resilience, and allows the clinician to view the contents of lumen 16. Additionally, a plastic product provides additional comfort to the patient by avoiding the “cold” feeling of metallic instruments. In the preferred embodiment, outer tube 11 has a length of about 20 cm to 30 cm and a diameter of about 3 mm to 5 mm.

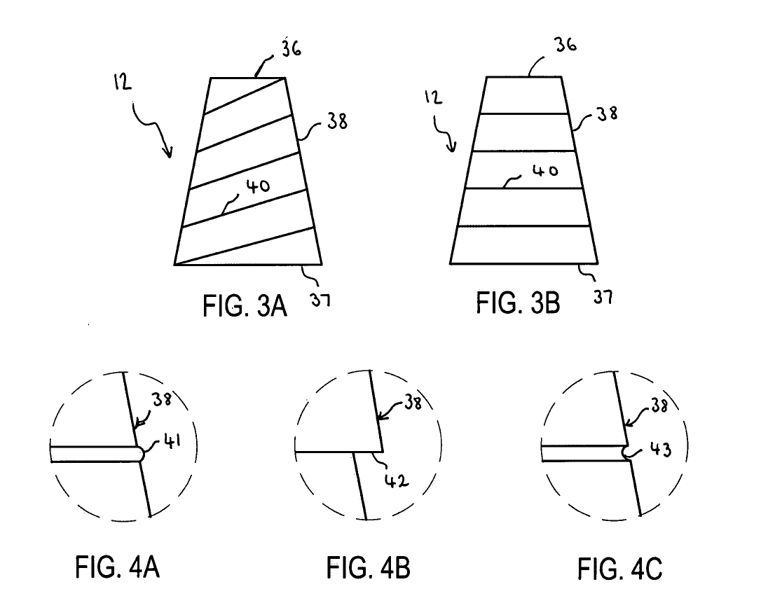

Referring now to FIGS. 3A-3B, applicator 12 preferably has a frustoconical shape, defined by distal base 36, proximal base 37, and lateral wall 38, and is dimensioned to conform as closely as possible to cervical anatomy. In a preferred embodiment, distal base 36 has a diameter of approximately 4 mm, proximal base 37 has a diameter of approximately 15 mm, later wall has a length of approximately 24 mm, and applicator 12 has a height of approximately 15 mm. Other embodiments may vary in size to accommodate different types of patients.

Applicator 12 is preferably manufactured from a resilient material, to provide for a soft contact and relative adaptation to the anatomical features of the patient while retaining its basic shape. In a preferred embodiment, applicator 12 is manufactured from a silicone material and is affixed to distal end 13 using a suitable method, for example, by the application of a biocompatible adhesive or by interference fit.

Referring more particularly to FIG. 3A, one or more ridges 40 are disposed along lateral wall 38, preferably in a helical pattern between distal base 36 and proximal base 37, as shown in FIG. 3A. Ridges 40 provide for applicator 12 to have a tighter fit with the cervical os, preventing the movement or dislodgment of applicator 12 during the injection of the anesthetic and the subsequent introduction of the catheter in the uterine cavity. Additionally, ridges 40 cause applicator 12 to essentially operate as a tenaculum by anchoring applicator 12 in the cervical os and by maintaining the cervical os in a dilated position, thereby providing the clinician with increased flexibility during the entire procedure.

Ridges 40 may be disposed on wall 38 in a variety of patterns. For instance, the helical pattern of FIG. 3A may vary in ridge density and angle, and different ridge patterns may be employed, for example, a parallel circular pattern, as illustrated in FIG. 3B.

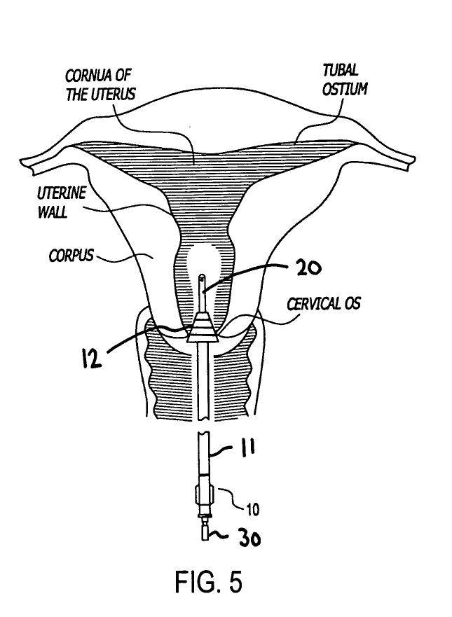

Referring now to FIGS. 4A-4C, ridges 40 may be configured in a variety of shapes. In the embodiment illustrated in FIG. 4A, ridges 40 are shaped like protrusions 41 that extend outwards of wall 38, providing a penetrating action into the walls of the cervical os and thus anchoring applicator 12 to the os. These protrusions preferably have a circular profile, but one of skill in the art will recognize that other protrusion shapes, for instance, multi-lateral protrusions are still within the spirit of the present invention.

In the embodiment illustrated in FIG. 4B, ridges 40 are defined by parallel layers of varying heights along wall 38 that are divided by scalloped edges 42 and that also provide a penetrating action into the walls of the cervical os, in a manner similar to ridges 41.

In the embodiment illustrated in FIG. 4C, ridges 40 are defined by grooves 43 that cause applicator 12 to have a screw-like shape, which adheres to the inner cervical walls with a rotational motion, providing a particularly strong anchoring effect.

Referring again to FIG. 2, inner tube 20 is preferably cylindrical, with a length that is at least 5 cm to 10 cm longer than outer tube 11 and with a diameter sufficient to permit inner tube 20 to be reciprocated inside lumen 16 and to extend beyond distal end 13, entering the uterine cavity. Inner tube 20 includes lumen 26 that extends between an opening 28 at proximal end 25 and one or more tissue collection apertures 22 disposed in the proximity of distal end 23. Although depicted in FIG. 2 as circular, apertures 22 may also be shaped according to other geometries or features to facilitate removal of a tissue sample, and may include flanges or angled edges. In a preferred embodiment, two apertures of semi-circular shape with a 3 mm diameter are disposed at 90 degrees from each other, beginning respectively at 2 and 7 mm from distal end 23.

Distal end 23 preferably has a rounded shape, to reduce the risk of accidental perforations of the uterine wall when distal end 23 contacts the uterine wall with excessive force. While distal end 23 is depicted as closed in FIGS. 1 and 2, in alternative embodiments distal end 23 may be removable by the clinician, thereby selectively allowing communication with lumen 26. Similarly, aperture 22 may be disposed at distal end 23, for example, where inner tube 20 further includes a pull wire or other means to articulate the distal region of inner tube 20.

In a preferred embodiment, inner tube 20 has markings 24 impressed on body 29 near proximal end 25 that enable the clinician to determine the depth of overall insertion into the uterus of inner tube 20 by measuring the translation of inner tube 20 in relation to proximal end 15.

Still referring to FIGS. 1 and 2, injection device 30 is configured to provide positive and negative pressures within lumen 26 of inner tube 20. Injection device 30 comprises shaft 39 connecting end portion 32 at distal end 33 with handle 34 at proximal end 35. End portion 32 preferably is configured so that its exterior surface is similar in shape to the interior surface of lumen 26 of inner tube 20, to maximize negative and positive pressures within lumen 16 when end portion 32 reciprocates within lumen 16.

Injection device 30 preferably comprises one or more pieces of a molded polymer, although other embodiments may have multiple pieces formed of different materials, such as a two-piece design with resilient piece 32 and rigid shaft 39. Injection device 30 preferably extends beyond the proximal end of inner tube 20 sufficiently far to allow a clinician to comfortably use handle 34 to manipulate injection device 30, for example, for a length of 2 cm to 3 cm beyond proximal end 15, although a shorter or longer length for injection device 30 may be desired in particular cases. Handle 34 may be simple in design, such as a continuation of shaft 39, or more complex, such as providing an increased diameter to facilitate grip, as depicted in FIGS. 1 and 2. Preferably, handle 34 is approximately 2 cm to 3 cm long and has a greater diameter than shaft 39, facilitating manipulation by the clinician.

When components 11, 20 and 30 are assembled, distal end 33 of injection device 30 is inserted into lumen 26, and distal end 23 of inner tube 20 is inserted into lumen 16. As depicted in FIG. 1, distal end 23 of inner tube 20 extends through opening 17 of outer tube 11. Markings 24 permit the clinician to determine how far inner tube 20 extends beyond the distal end of outer tube 11, and may include indications of the radial orientation of aperture 22 in relation to proximal end 25.

In a preferred embodiment, injection device 30 is a syringe that is suitable for connection to Luer-Lok fitting 14 for injecting an anesthetic medication, and for a later connection to inner tube 20 for drawing cell samples. Alternatively, two different syringes may be employed, one to be connected to Luer-Lok fitting 14 for injecting the anesthetic medication, and the other to be connected to inner tube 20 for drawing the cell samples.

Referring now to FIG. 5, a preferred method of using device 10 is described. First, the clinician inserts distal end 13 of outer tube 11 carrying applicator 12 into the patient's vaginal canal. The clinician then advances outer tube 11 and wedges applicator 12 into the patient's cervical os, either by direct pressure, or with a movement that is related to the disposition and orientation of ridges 40. The clinician's mode of insertion of applicator 12 is directed at achieving the most effective anchoring of applicator 12 to the cervical os while minimizing discomfort to the patient.

Once applicator 12 is so positioned, the clinician may attach a conventional syringe containing an anesthetic (for example, lidocaine) to Luer-Lok fitting 14 and deliver the desired dosage of anesthetic, which passes through lumen 16 into the cervical canal and the uterine cavity. The tight contact of applicator 12 with the cervical os reduces leakage of the anesthetic into the vaginal space by acting as a physical barrier.

Once sufficient time has passed for the anesthetic to take effect, the clinician removes the syringe from Luer-Lok fitting 14 and inserts distal end 23 of inner tube 20 into opening 18. Inner tube 20 is connected at proximal end 25 with a syringe of dimensions adequate to draw vacuum and cell samples from the uterus. The clinician may refer to markings 24 and turn inner tube 20 so that aperture 22 is facing in the desired direction.

Inner tube 20 and injection device 30 are advanced through lumen 16 until distal end 23 of inner tube 20 extends beyond the distal end of outer tube 11 and penetrates into the patient's uterus. The clinician then draws vacuum, causing the endometrial tunic of the uterus to release cells that are suctioned into aperture 22 for collection.

During the entire procedure, applicator 12 remains positioned and anchored in the cervical os, maintaining the cervical os constantly dilated like a tenaculum and facilitating the activity of the clinician. At the end of the procedure, applicator 12 is disengaged from the cervical os by a suitable action of the clinician, and the outer and inner tubes are removed from the patient's vaginal canal.

Although preferred illustrative embodiments of the present invention are described hereinabove, it will be evident to one skilled in the art that various changes and modifications may be made therein without departing from the invention. It is intended in the appended claims to cover all such changes and modifications that fall within the true spirit and scope of the invention.

What is claimed is:

1. An apparatus for performing an endometrial biopsy on a patient, the apparatus comprising:
an outer member having a distal end, a proximal end and a lumen extending therebetween;
an applicator surrounding the distal end of the outer member and sized to engage the cervical os of the patient, the applicator comprising an outer surface having one or more ridges;
an inner member having a distal end including one or more collection openings, a proximal end and a lumen extending between the one or more collection openings and the distal end, the inner member being configured to reciprocate within the lumen of the outer member and to extend beyond the distal end of the outer member and into the uterine cavity; and
an injection device for injecting and drawing fluid and tissue through the one or more collection openings and the lumen of the inner member.

2. The apparatus of claim 1, wherein the outer member further comprises a Luer-Lok fitting disposed at its proximal end.

3. The apparatus of claim 1, wherein the applicator has a frustoconical shape.

4. The apparatus of claim 1, wherein the one or more ridges are disposed around the longitudinal axis of the applicator in a helical pattern.

5. The apparatus of claim 1, wherein the one or more ridges are circularly disposed around the longitudinal axis of the applicator.

6. The apparatus of claim 1, wherein the one or more ridges are defined by grooves carved on the outer surface of the applicator.

7. The apparatus of claim 1, wherein the one or more ridges are defined by layers of varying heights.

8. The apparatus of claim 1, wherein the one or more ridges are defined by protrusions projecting from the outer surface of the applicator.

9. The apparatus of claim 1, wherein the applicator is made of a resilient material.

10. The apparatus of claim 9, wherein the applicator is made of a silicone material.

11. The apparatus of claim 1, wherein the inner member further comprises one or more markings near the proximal end.

12. The apparatus of claim 1, wherein the injection device comprises a plunger configured to reciprocate through the lumen of the inner member.

13. The apparatus of claim 1, wherein the injection device comprises a syringe coupled to the proximal end of the inner member via a Luer-Lok fitting.

14. The apparatus of claim 1, wherein the inner member has a length in the range of 5 to 10 cm longer than the length of the outer member.

15. The apparatus of claim 1, wherein the outer and inner members are formed from a semi-rigid polymeric material.

16. A method for performing an endometrial biopsy comprising:
providing an apparatus having an outer member, an applicator, an inner member, and an injection device, the outer member having a distal end, a proximal end and a lumen extending therebetween, the applicator the distal end of the outer member, the applicator further comprising an outer surface having one or more ridges, the inner member having a distal end including one or more collection openings, a proximal end, and a lumen extending between the one or more collection openings and the proximal end, the inner member being configured to reciprocate within the lumen of the outer member and to extend beyond the distal end of the outer member and into the uterine cavity, the inner member further having an opening disposed in the proximity of its distal end;
inserting the distal end of the outer member into the genital system of a female patient;
anchoring the applicator in the cervical os of the patient, the applicator holding the cervical os in a dilated position;
delivering medication through the lumen of the outer member;
inserting the distal end of the inner member beyond the distal end of the outer member;
applying suction to the proximal end of the inner member to draw cells from the patient through the opening in the inner member; and
removing the apparatus from the patient by de-anchoring the applicator from the cervical os.

17. The method of claim 16, wherein the applicator has a frustoconical shape, and wherein the applicator is anchored in the cervical os by pressure contact between at least some of the one or more ridges and the cervical os.

18. The method of claim 16, wherein suction is applied by applying negative pressure with a syringe coupled to the proximal end of the inner member.

19. The method of claim 16, wherein the medication is an anesthetic.

20. The method of claim 20, wherein the anesthetic is lidocaine.

21. The method of claim 16, wherein the applicator is de-anchored by rotating the outer tube.

22. The method of claim 16, wherein the applicator is de-anchored by twisting and pulling on the outer tube.
